(19) United States
(12) Patent Application Publication
(10) Pub. No.: US 2018/0147033 A1
(43) Pub. Date: May 31, 2018

(54) POLYARYLETHERKETONE DENTAL BLOCK FOR CAD/CAM MILLING

(71) Applicant: JUVORA LIMITED, Lancashire (GB)

(72) Inventors: Nuno SERENO, Southport Merseyside (GB); Marcus JARMAN-SMITH, Lytham St. Annes Merseyside (GB); Reinhard LOBENHOFER, Nuernberg (DE)

(73) Assignee: JUVORA LIMITED, Lancashire, OT (GB)

(21) Appl. No.: 15/106,100

(22) PCT Filed: Dec. 17, 2014
(86) PCT No.: PCT/GB2014/053728

§ 371 Date: Jun. 17, 2016

(30) Foreign Application Priority Data
Dec. 19, 2013 (GB) 1322565.1

(51) Int. Cl. A61C 13/00; A61K 6/087; A61C 5/77; A61K 6/033

(52) U.S. Cl. CPC A61C 13/0022; A61K 6/033; A61C 5/77; A61K 6/087

(57) ABSTRACT

A method of making a prosthetic dental item comprises selecting a block having a square or rectangular cross-section along is extent, wherein said block comprises polyetheretherketone and, optionally, an apatite, wherein said polymeric material has a crystallinity of at least 25%. The method comprises machining the block in dependence upon data collated using digital technology. Since the crystallinity of the material selected is high, a post-machining step whereby crystallinity is increased can be avoided, and dental items of high precision can be formed.

The invention relates to prosthetic dental items and particularly, although not exclusively, relates to a block for use in the manufacture of prosthetic dental items, for example dental restorations, for example using CAD/CAM milling.

Conventionally, a dental restoration procedure that includes creating a prosthetic dental item is a laborious and time-consuming “trial and error” process. A dentist can create a dental mould of the patient's teeth and gums by capturing a physical impression of the patient's teeth and gums using a moulding material. The dental mould is forwarded to a dental laboratory where a physical, three-dimensional model of the patients teeth and gums is created.

At the dental laboratory, a technician pours plaster into the mould. Once the plaster dries and is removed from the mould, the moulded plaster is used as a physical, three-dimensional model of the patient's teeth and gums. If a dental restoration procedure includes replacing a missing tooth with a prosthetic tooth, the technician can build a wax model of the missing tooth using the plaster model of the patients teeth and gums. The wax model can be used to cast a metal framework to which porcelain will be adhered. The technician adjusts colouring of the porcelain and fires the porcelain and the metal framework in a furnace to bake the porcelain onto the metal framework to create the prosthetic tooth. The technician can add several additional layers of porcelain to the prosthetic tooth to simulate natural colour properties (e.g., hue, saturation, and chrominance) of the patients missing tooth. Once the prosthetic tooth is finished, the technician returns the prosthetic tooth to a dentist, who examines it, and occasionally returns it to the laboratory for re-working if problems with the colour properties or dimensions of the prosthetic tooth are discovered.

More recently, computer-aided design (CAD) and computer-aided manufacturing (CAM) technologies have been used to create prosthetic dental items. CAD/CAM technologies can be employed to produce prosthetic dental items that fit into a patient's mouth more precisely compared to prosthetic dental items built using conventional techniques. In addition, prosthetic dental items built using CAD/CAM technologies can be produced more quickly than with conventional techniques. For example, Sirona Dental Systems produces a Cerec® system that can be used in a dentist's practice (e.g. in “chair-side CAD/CAM restoration wherein a restoration is produced in a dentist's practice within hours and is designed, produced and fitted in a patient's mouth during a single visit to the practice).

Current dental CAD/CAM systems for chair-side CAD/CAM restorations may employ a ceramic block which has a square or rectangular cross-section and is milled by a milling machine at the dentist's practice to produce a prosthetic dental item, for example an inlay, onlay, crown, bridge or abutment.

One commercially available ceramic block for chair-side use is IPSe.max CAD which is a lithium disilicate glass-ceramic block. It is produced and initially processed in a CAD/CAM machine in a crystalline intermediate state to produce a milled prosthetic dental item. After milling, it is crystallised in a furnace at high temperature (e.g. 840-850° C.) over a period of time. However, such a process can be relatively time-consuming and thereby delay completion of the chair-side restoration. Furthermore, the crystallization process may lead to shrinkage of the dental item which may affect how well it fits within a patient's mouth.

It is an object of the present invention to provide a block for use in manufacturing a prosthetic dental item which may be advantageous over prior art blocks.

It is an object of the present invention to provide a block for use in manufacturing a prosthetic dental item which need not be subjected to a crystallisation process after milling.

According to a first aspect of the invention, there is provided a method of making a prosthetic dental item, the method comprising:
(i) selecting a block having a square or rectangular cross-section along is extent, wherein said block comprises a polymeric material which comprises a repeat unit of formula (I):

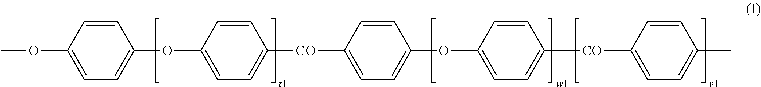

where t1 and w1 independently represent 0 or 1 and v1 represents 0, 1 or 2;
wherein said polymeric material has a crystallinity of at least 10%;
(ii) machining the block in dependence upon data collated using digital technology.

Advantageously, since the crystallinity of the material selected is high, a post-machining step whereby crystallinity is increased can be avoided, thereby saving time and avoiding the need for a means for increasing crystallinity (e.g. a furnace as in the prior art) to be available for use.

The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning calorimetry (DSC). Preferably, crystallinity is measured as described in Example 2.

In especially preferred embodiments, the crystallinity of said polymeric material may be greater than 20% or greater, than 25%. The crystallinity is, especially, greater than 30%. It may be less than 50% or less than 40%. Preferably the dental item includes a framework having the aforementioned levels of crystallinity.

Said block may have a volume of at least 900 mm³. The volume may be less than 65,000 mm³. The volume is preferably in the range 1,500 to 16,000 mm³. The smaller volume blocks may be used for a single crown or single abutment; the largest may be used for bridges with four or more units or combined long implant/abutment pieces.

Said block may have a minimum side length of 7 mm and a maximum of 75 mm. Said block may have a cross-section with an area in the range 50 mm² to 1000 mm² (preferably 50 to 200 mm²). Said block may have a length in the range 10 mm to 80 mm (preferably 15 mm to 20 mm).

Said polymeric material preferably consists essentially of a repeat unit of formula I. Preferred polymeric materials comprise (especially consist essentially of) a said repeat unit wherein t1=1, v1=0 and w1=0; t1=0, v1=0 and w1=0; t1=0, w1=1, v1=2; or t1=0, v1=1 and w1=0. More preferred comprise (especially consist essentially of) a said repeat unit wherein t1=1, v1=0 and w1=0; or t1=0, v1=0 and w1=0. The most preferred comprises (especially consists essentially of) a said repeat unit wherein t1=1, v1=0 and w1=0.

In preferred embodiments, said polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said polymeric material may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². Said Notched Izod Impact Strength, measured as aforesaid, may be less than 10 KJm⁻², suitably less than 8 KJm⁻². The Notched Izod Impact Strength, measured as aforesaid, may be at least 3 KJm⁻², suitably at least 4 KJm⁻², preferably at least 5 KJm⁻². Said impact strength may be less than 50 KJm⁻², suitably less than 30 KJm⁻².

Said polymeric material suitably has a melt viscosity (MV) of at least 0.06 KNsm⁻², preferably has a MV of at least 0.09 KNsm⁻², more preferably at least 0.12 KNsm⁻², especially at least 0.15 KNsm⁻². Advantageously, the MV may be at least 0.35 KNsm⁻² and especially at least 0.40 KNsm⁻². An MV of at least 0.40 KNsm⁻² (e.g. 0.40 to 0.50 KNsm-²) has been found to be particularly advantageous in the manufacture of accurate, strong frameworks.

MV is suitably measured using capillary rheometry operating at 400° C. at a shear rate of 1000 s⁻¹ using a tungsten carbide die, 0.5 mm×3.175 mm.

Said polymeric material may have a MV of less than 1.00 KNsm⁻², preferably less than 0.5 KNsm⁻².

Said polymeric material may have a MV in the range 0.09 to 0.5 KNsm⁻², preferably in the range 0.14 to 0.5 KNsm⁻², more preferably in the range 0.4 to 0.5 KNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

The main peak of the melting endotherm (Tm) of said polymeric material may be at least 300° C.

Said block may be made from a composition which includes said polymeric material described and other components, for example colourants (e.g. pigments, ceramics, metal oxides (eg. titanium dioxide)) or fillers (for example radiopaque fillers such as barium sulphate). Said composition may include 0-10 wt %, suitably 0-6 wt % of colourants. Colourants may be selected so the composition is pink or white. In one embodiment, the composition includes no colourant.

In a first preferred embodiment (e.g. wherein the dental item is not osseointegrated in use), said composition comprises at least 80 wt %, at least 90 wt % or at least 94 wt % of said polymeric material. The balance may comprise one or more colourants. In an especially preferred embodiment, said composition comprises at least 99 wt % of said polymeric material, especially polyetheretherketone.

In a second preferred embodiment, said block comprises a composition comprising said polymeric material (especially polyetheretherketone and/or a polymeric material consisting essentially of repeat units of formula I wherein t1=1, w1=0 and v1=0) and an apatite, for example a hydroxyl-containing apatite (especially hydroxyapatite). The ratio of the wt % of said polymeric material divided by the wt % of said apatite may be in the range 1 to 9.

The ratio of the wt % of said polymeric material divided by the wt % of said apatite may be in the range 2.3 to 9, is suitably in the range 2.7 to 5.6, preferably in the range 3 to 5, more preferably in the range 3.5 to 4.5, especially in the range 3.8 to 4.2 or 3.9 to 4.1.

Said composition suitably includes at least 65 wt %, preferably at least 70 wt %, more preferably at least 75 wt % of said polymeric material, and may include at least 10 wt %, preferably at least 15 wt %, more preferably at least 18 wt % of said apatite. The balance in said composition may be made up of other fillers, for example colourants. Preferably, the sum of the wt % of said polymeric material and said apatite is in the range 90 to 100 wt %, more preferably in the range 95 to 100 wt %, especially 99 to 100 wt %.

Said apatite may optionally comprise a material that has been modified or doped with one or more additional chemical elements. For example, it may comprise a material that has been modified or doped with one or more metals. The apatite may for example comprise hydroxyapatite that has optionally been modified or doped. The hydroxyapatite may for example optionally be modified or doped with one or more metals. The hydroxyapatite may for example be optionally modified or doped with boron, magnesium, silicate or silver.

The apatite may comprise a material optionally doped with one or more of silicate (SiO₄²), Borate (BO₃³⁻) and Strontium (Sr²⁺). Suitably, the total content of silicate (SiO₄²⁻), Borate (BO₃³⁻) and Strontium (Sr²⁺) within the apatite does not exceed 10% by molarity as a cumulative value.

Said apatite may comprise one or more of Silicon (Si), Fluorine (F), Sulphur (S), Boron (B), Strontium (Sr), Magnesium (Mg), Silver (Ag), Barium (Ba), Zinc (Zn), Sodium (Na), Potassium (K), Aluminium (Al), Titanium (Ti) and Copper (Cu).

Said apatite may comprise a material comprising a calcium phosphate lattice, for example a hydroxyapatite lattice in which, optionally, single or multiple elements have been introduced. For example the apatite may comprise a calcium phosphate lattice into which, optionally, one or more of Silicon (Si), Fluorine (F), Sulphur (S), Boron (B), Strontium (Sr), Magnesium (Mg), Silver (Ag), Barium (Ba), Zinc (Zn), Sodium (Na), Potassium (K), Aluminium (Al), Titanium (Ti) and Copper (Cu) have been introduced.

Said apatite is preferably a hydroxyapatite. Said apatite is preferably hydroxyapatite. Suitably, 90 to 100 wt %, preferably 95 to 100 wt %, preferably 98 to 100 wt % of said apatite is made up of calcium, phosphorous, oxygen and hydrogen moieties. Said apatite is preferably a hydroxyapatite which consists essentially of calcium, phosphorous, oxygen and hydrogen moieties.

The D50 of said apatite, assessed using laser diffraction and based on a volume distribution, is suitably less than 200 μm, preferably less than 100 μm, more preferably less than 50 μm, especially less than 20 μm. The D50 may be at least 0.1 μm, preferably at least 0.51 μm, more preferably at least 1.0 μm.

In the method, in a step (i)* after step (i) and before step (ii), preferably digital technology is used to collate data on the region into which the dental item is to fit. Step (i)* preferably includes scanning a region into which the dental item is to fit (e.g. scanning a patient's mouth) or scanning of a model of a region into which the dental item is to fit. Preferably, data is collated from a model, for example a cast, obtained of part of a patient's mouth and/or dentition. Step (i)* may comprise use of Computer-aided design (CAD) technology.

The method preferably includes a step prior to step (i)* of taking an impression of at least part of a patient's mouth. The impression may be used to collate said data.

The method preferably involves a CAD/CAM technique whereby data is obtained (e.g. from scanning a patient's mouth or a model thereof) and computer-aided manufacture (CAM) is undertaken in step (ii). Thus, in step (ii), a computer suitably controls the machining of the block.

Preferably, the selected block is positioned in a machine (e.g. a milling machine) and the machine is arranged to machine the block in dependence upon the data.

Preferably, machining of said block is undertaken using at least a 3-axis machine, but preferably a 3+2 axis or 5 axis machine, suitably under computer control. Machining in step (ii) suitably comprises milling. The work piece is suitably cooled during machining so as to achieve a good surface finish and preserve the milling tools.

Preferably, after step (ii), a dental item is produced which includes no metal and preferably consists essentially of material derived from said block.

Using the method, a dental item of great accuracy can be made which can be fitted in position in a patient's mouth with no need for alteration. The manufacturing accuracy is facilitated by producing highly crystalline dental items as described. Preferably, the ratio of the crystallinity (measured by DSC) of the block selected in step (i) divided by the crystallinity (measured by DSC) of the dental item produced in the method is in the range 0.8 to 1.2, more preferably in the range 0.9 to 1.1, especially about 1.

Said dental item made in the method preferably includes an area of thickness less than 2 mm. Said dental item, for example a framework, preferably includes an area of at least 0.5 cm², preferably at least 1 cm² which has a thickness of less than 2 mm.

Said dental item preferably includes an area of thickness less than 1.5 mm. Said dental item preferably includes an area of at least 0.2 cm², preferably at least 1 cm² which has a thickness of less than 1.5 mm. Said dental item preferably includes an area of at least 0.5 cm², preferably at least 1 cm² which has a thickness of less than 1.0 mm.

Said dental item may be selected from an inlay, onlay, crown, bridge, dental implant or abutment.

In one embodiment, wherein said dental item is osseointegrated in use (e.g. it is an implant), said item may be made from said block which comprises said polymeric material and an apatite as described in detail above.

According to the second aspect of the invention, there is provided a dental item made in the method of the first aspect per se.

The dental item may have any feature of the dental item described according to the first aspect.

According to a third aspect of the invention, there is provided a block for use in the method of the first aspect, said block consisting essentially of a composition which includes:

(a) at least 99 wt % of a polymeric material wherein said polymeric material comprises a repeat unit of formula (I)

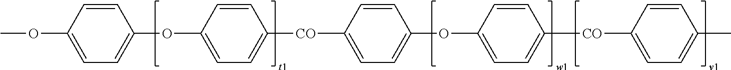

where t1 and w1 independently represent 0 or 1 and v1 represents 0, 1 or 2, wherein said polymeric material has a crystallinity measured by DSC of at least 10% (preferably 20 to 36%); or
(b) said polymeric material of formula I and an apatite, said polymeric material having t1, w1, v1 and said crystallinity as described in (a), wherein the ratio of the wt % of said polymeric material divided by the wt % of said apatite is in the range 1. to 9.

Said block, said composition and said polymeric material may have any features described in the other aspects

Any invention described herein may be combined with any feature of any other aspect of any other invention or embodiment described herein mutatis mutandis.

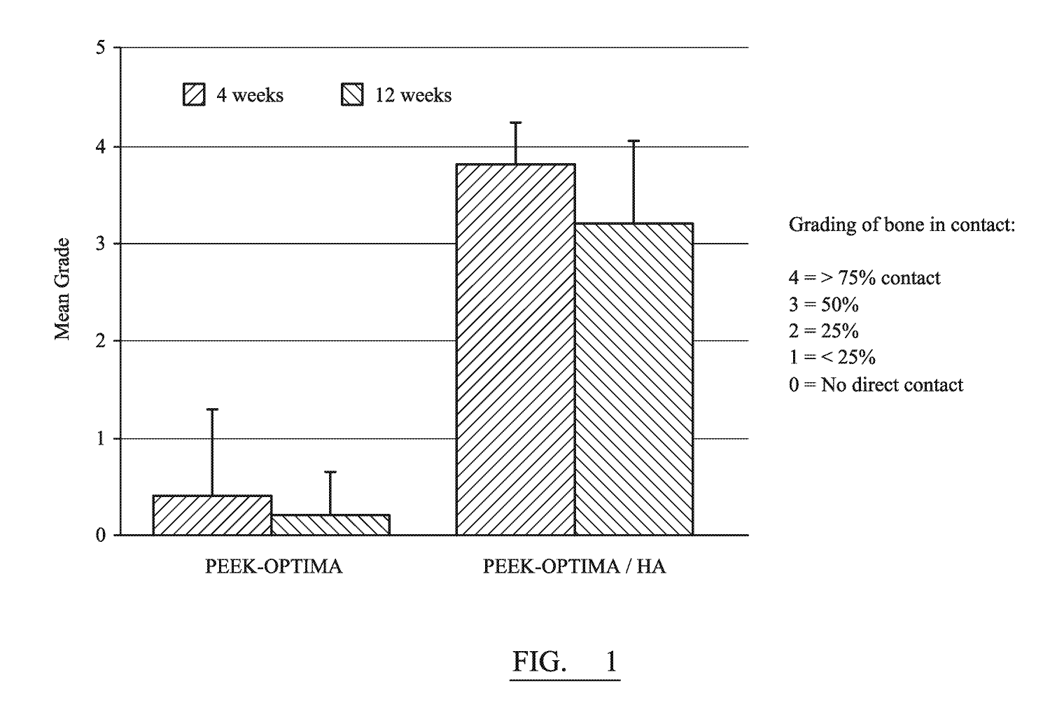

Specific embodiments of the invention will now be described, by way of example, with reference to FIG. 1 which is a graph showing the results of grading of bone.

### EXAMPLE 1—MANUFACTURE OF POLYETHERETHERKETONE (PEEK) BLOCK

Blocks may be made having the following dimensions by injection moulding: 8×8×15 mm, 8×10×15 mm, 10×12×15 mm, 12×14×18 mm, 14×14×18 mm, 15×21×40 mm, 15×21×50 mm, 15×21×70 mm, 25×40×60 mm.

The blocks may be made by Process 1 or Process 2.

Process 1. Extrusion:

Step 1—A PEEK rod is produced by the means of extrusion.

Step 2—The PEEK rod is annealed, if required. The crystallinity might rise to 35% during annealing.

Step 3—A section of the PEEK rod is milled to the size and shape of the final PEEK block. (PEEK Block average crystallinity would be similar to PEEK rod average crystallinity). The crystallinity of the block will be substantially the same as the rod used in its manufacture.

Process 2. Injection Moulding:

Step 1—A PEEK block is produced by means of injection moulding or compression moulding.

Step 2—The PEEK block is annealed. The crystallinity might raise to 35% during annealing

### EXAMPLE 2—MEASUREMENT OF CRYSTALLINITY

The level and extent of crystallinity in a polymer may be measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning calorimetry (DSC) in a process such as the following which is also described in POLYMER Vol. 37, Number 20, 1996, page 4573.

DSC may be used to examine a 10 mg plus or minus 10 microgram sample of polymeric material in a TA Instruments DSC Q100 under nitrogen at a flow rate of 40 ml/min. The scan procedure may be:
Step 1 Perform and record a preliminary thermal cycle by heating the sample from 30° C. to 450° C. at 20° C./min, recording the Tg, Tn and Tm.
Step 2 Hold for 2 mins
Step 3 Cool at 10° C./min to 30° C. and hold for 5 mins, recording Tc.

Step 4 Heat from 30° C. to 450° C. at 20° C./min, recording the Tg and Tm.

From the resulting curve the onset of the Tg may be obtained as the intersection of lines drawn along the pre-transition baseline and a line drawn along the greatest slope obtained during the transition. The Tn is the temperature at which the main peak of the cold crystallisation exotherm reaches a maximum. The Tm is the temperature at which the main peak of the melting endotherm reaches a maximum. The Tc is the temperature at which the main peak of the crystallisation from the melt exotherm reaches a maximum.

The Heat of Fusion (ΔH (J/g)) may be obtained by connecting the two points at which the melting endotherm deviates from the relatively straight baseline. The integrated area under the endotherm as a function of time yields the enthalpy (mJ) of the transition, the mass normalised Heat of Fusion is calculated by dividing the enthalpy by the mass of the specimen (J/g). The level of crystallisation (%) is determined by dividing the Heat of Fusion of the specimen by the Heat of Fusion of a totally crystalline polymer, which for polyetheretherketone is 130 J/g.

The aforementioned methods provide the level of crystallinity in a bulk sample. As an alternative, FTIR may be used to assess crystallinity and this may be used to assess the level of crystallinity at a surface and/or across the thickness or surface of a sample. Reference is made to a paper titled “Crystallinity in Poly(Aryl-Ether-Ketone) Plaques Studied by Multiple Internal Reflection Spectroscopy” (Polymer Bull, 11, 433 (1984)).

In a preferred embodiment, DSC may be used to measure crystallinity of a bulk sample. FTIR may be used to measure crystallinity at a surface.

### EXAMPLE 3—MANUFACTURE OF PROSTHODONTICS DENTAL ITEM (E.G. INLAY, ONLAY, CROWN, BRIDGE OR ABUTMENT)

The following steps are undertaken:

(i) A mould is taken of a patient's mouth using a standard impression tray. The mould is then poured with dental plaster and allowed to set.

(ii) The mould is scanned to collate relevant CAD data which is input into a CAD/CAM milling machine (e.g. a Cerec or Sirona machine). An operator then designs the dental item in conjunction with the machine. The machine is suitably set up to prepare a CAD design for the manufacture of the item from a PEEK block made in Example 1.

(iii) A PEEK block is inserted in the CAD/CAM machine which operates automatically to machine the item from the block, based on data collated from scanning the mould.

(iv) The framework is removed from the block and finished as required.

As an alternative to use of a mould as described, a digital scan of a relevant part of the patient's mouth could be taken and relevant digital data input into the CAD/CAM machine.

The blocks used in Example 1 may consist exclusively of PEEK and therefore take on the natural colour of the PEEK or alternatively the PEEK may incorporate one or more fillers to render the block (and the dental item made therefrom), for example pink, white or gold. Alternatively or additionally, a dental veneer may be applied to a dental item made as described.

Advantageously, since the block used to form the dental item has a high level of crystallinity, it does not need to be treated to increase or adjust its crystallinity. Thus, a dental item can be produced rapidly in a “chair-side” procedure.

As an alternative to provision of PEEK blocks, blocks incorporating hydroxyapatite may be provided. These may be particularly advantageous for making dental implants since the PEEK-HA material described has been found to have improved bioactivity as illustrated below.

### EXAMPLE 4—MANUFACTURE OF COMPOSITION COMPRISING POLYETHERETHERKETONE (PEEK) AND HYDROXYAPATITE (HA)

Polyetheretherketone (PEEK) obtained in the form of PEEK-OPTIMA® LTI (Invibio Biomaterial Solutions, UK) having a melt viscosity (MV) of 0.44 KNsm⁻² was dried to remove water (it absorbs water during storage). The PEEK was in the form of granules of approximately 3 mm by 2 mm size. Hydroxyapatite (HA) in the form of particles having mean particle size of about 5 μm was selected.

The PEEK and HA were mixed in a twin screw compounder (extruder) which heated the mixture to between 360° C. and 400° C. (with a temperature of 400° C. at the extruder output) to melt the PEEK. The PEEK was introduced to the extruder at a point upstream from the introduction of HA to the extruder. The PEEK was heated and conveyed through the extruder such that the PEEK was in a molten state within the extruder before the HA was added. The mixture of HA and molten PEEK was then conveyed further through the extruder to mix the PEEK and HA. A PEEK and HA composite was extruded from the extruder and pelletized.

The PEEK and HA were added to the extruder in a ratio such that the output of the extruder was a PEEK and HA composite which comprised 10 wt % of HA.

The extruder comprised a normal screw profile fabricated from stainless steel with a minimum L/D ratio of 45:1. At the extrusion end a twin hole die with a 4 mm orifice and pelletizer was used. The main screw rotation speed was set at 150-250 rpm. The screws were intermeshing counter-rotating screws having a length of around 1 m and a diameter of around 40 mm. Laces of approximately 2 mm diameter were chopped to lengths of approximately 3 mm to define the PEEK and HA composite pellets.

### EXAMPLES 5—GENERAL PROCEDURE FOR MAKING INJECTION MOULDED COMPONENTS

Pellets (e.g. those of Example 4) were injection moulded to produce a bioactive component. An injection moulding machine used comprised a heated barrel through which the pellets were conveyed by a screw. The barrel was heated to temperatures of between 360° C. and 375° C. such that the polymeric material within the pellets melted as they were conveyed through the barrel such that a melt was produced. The melt was then injected through a nozzle into a mould with the mould tool being heated to between 200° C. and 220° C.

Mechanical properties, including Izod impact strength (Notched) (ISO 180), flexural strength (ISO 178), flexural modulus (ISO 178), tensile strength (ISO 527), and strain at break (ISO 527) of a test specimen were determined and the results are shown in Table 1.

### EXAMPLES 6 TO 9

The method of Example 4 was repeated but the ratio of PEEK to HA was adapted such that the output of the extruder was a PEEK and HA composite which comprised a different wt % of HA, as detailed in Table 1.

TABLE 1

| Example No. | PEEK (wt %) | HA (wt %) |
|---|---|---|
| 6 | 80 | 20 |
| 7 | 70 | 30 |
| 8 | 60 | 40 |
| 9 | 50 | 50 |

The PEEK and HA composite pellets produced were injection moulded as described in Example 5 to produce bioactive components. Mechanical properties of components made were determined and the results are shown in Table 2.

### COMPARATIVE EXAMPLE 1

Polyetheretherketone (PEEK) obtained in the form of PEEK-OPTIMA® (Invibio Biomaterial Solutions, UK) was used in an injection moulding machine and injection moulded to produce a component following the general procedure of Example 5. Mechanical properties were determined for comparison with the components of Examples 4 and 6 to 9 and the results are shown in Table 2.

### Results

The results of the mechanical tests are detailed in Table 2 below

TABLE 2

| | Comparative | | | | | |
|---|---|---|---|---|---|---|
| | Example | Example 4 | Example 6 | Example 7 | Example 8 | Example 9 |
| Property | (No HA) | (10% HA) | (20% HA) | (30% HA) | (40% HA) | (50% HA) |
| Impact | 7.33 | 7.4 | 6.1 | 5.2 | 4.6 | 4.6 |
| Strength | | | | | | |
| (KJ/m2) | | | | | | |
| Flexural | 162.45 | 156.1 | 156.0 | 154.2 | 139.2 | 118.8 |
| strength | | | | | | |
| (MPa) | | | | | | |
| Flexural | 3.96 | 4.33 | 4.72 | 5.61 | 6.67 | 8.02 |
| modulus | | | | | | |
| (GPa) | | | | | | |
| Tensile | 99.25 | 88.7 | 88.7 | 81.8 | 73.5 | 75.5 |
| Strength | | | | | | |
| (MPa) | | | | | | |
| Strain at | 35.8 | 24.09 | 8.8 | 3.98 | 2.24 | 1.27 |
| Break (%) | | | | | | |

It was found that PEEK could be successfully compounded with HA up to 50 wt % HA, without significant difficulties and with no reaction observed between the two components. The mean mechanical values for impact strength, flexural strength, flexural modulus, tensile strength, and strain at break were plotted (plots not shown) against the filler content and compared with those of the unfilled PEEK to determine optimum HA levels. From this it was concluded that 20 wt % of HA (Example 6) gave the optimum level to allow HA to be present at sufficient levels to provide desirable bioactivity to the component without significant detriment to the physical properties

### EXAMPLE 10—BIOACTIVITY TESTS

PEEK containing 20% by weight HA (Example 6) was chosen for further bioactivity studies due to the limited effects on material mechanical properties compared to PEEK alone (Comparative Example 1).

Bioactivity of the PEEK/HA was determined by the ability to form apatite on the surface of the material in a simulated body fluid (SBF) using SBF-JL2 as prepared and described in Bohner and Lemaitre (Bohner M, Lemaitre J./Biomaterials 30 (2009) 2175-2179) and compared with controls comprising PEEK alone.

The SBF-JL2 was produced using a dual-solution preparation (Sol. A and Sol.B) having the following composition for 2 litres of final fluid:

| | | | | |
|---|---|---|---|---|
| Starting Materials | MW | Purity | | |
| Formula | [g/mol] | [—] | Sol. A | Sol. B |

Weights of starting
materials [g/L]

| | | | | |
|---|---|---|---|---|
| NaCl | 58.44 | 99.5% | 6.129 | 6.129 |
| NaHCO₃ | 84.01 | 99.5% | 5.890 | |
| Na₂HPO₄•2H₂O | 177.99 | 99.0% | 0.498 | |
| CaCl₂ | 110.99 | 95.0% | | 0.540 |

Volume of HCl
solution (mL/L)

| | | | | |
|---|---|---|---|---|
| HCl 1.00M | Aq. Sol. | [mL/L] | 0.934 | 0.934 |

Use of this in vitro method of examining apatite formation as a means of predicting in vivo bone bioactivity is both widely used and accepted (Kokubo T, Takadama H. How useful is SBF in predicting in vivo bone bioactivity? Biomaterials 2006; 27(15):2907-2915). Samples were immersed in SBF for 1, 3 and 7 days on a rotating platform at 37° C. with 5% CO₂ and 100% humidity. X-ray photoelectron spectroscopy (XPS), scanning electron microscopy (SEM), and attenuated total reflectance Fourier transform infrared spectroscopy (ATR-FTIR) were used to analyse the bioactive elements present on the surface of the specimens following immersion in SBF.

SEM analysis of the surface of PEEK controls and PEEK/20% HA composite revealed the formation of spherical crystals on the surface after immersion in SBF. These were more numerous and apparent on the PEEK/20% HA samples and these were observed as early as 1 day post-immersion in SBF, suggesting increased apatite formation.

Detailed Ca2p and P2p XPS spectra revealed that although Ca and P were identified on the surface of both materials, only elemental ratios present on the PEEK/20% HA samples were conducive to bone formation with a Ca/P ratio of 1.66, close to the theoretical value for hydroxyapatite. Meanwhile, the ratios of the depositions on the control PEEK were more variable (>1.67), and indicative of non-hydroxyapatite calcium phosphate formations.

Following immersion in SBF for 1 day, ATR-FTIR surface analysis was performed on PEEK/20% HA and control PEEK samples to semi-quantify the degree of apatite deposition and detect functional groups. A significant peak was observed at 1015 cm⁻¹, most likely arising from the structural P—O bond of phosphate groups. The ratio of absorption at 1015 cm⁻¹ to 1645 cm⁻¹ (characteristic of PEEK) was measured and showed an increased ratio on PEEK/20% HA samples compared with control PEEK, confirming the XPS findings indicating greater apatite formation on the PEEK/20% HA samples.

Surprisingly it has been found that despite the low proportion of HA in the component (only 20% by weight) sufficient HA is available at the surface of the component to impart bioactive properties to the component and promote apatite formation.

### EXAMPLE 11—ASSESSMENT OF DEGREE OF DIRECT IMPLANT-BONE CONTACT IN AN OVINE PRE-CLINICAL STUDY

Cylindrical dowels of the composition of Example 6 and PEEK-OPTIMA were implanted in an established ovine model. Implants were placed in sheep tibia cortical bone for 4 weeks and 12 weeks. At the end of each time point, implants and surrounding bone were harvested and embedded in PMMA. Tissue sections were stained for histology using methylene blue and basic fuchsin. Histology images were graded on a semi-quantitative scale by two blinded observers to determine the percent bone ongrowth. At both the 4 week and 12 week time points, the percentage of direct bone contact was higher with the composition of Example 6 compared with PEEK-OPTIMA alone.

Results are provided in FIG. 1 which shows the grading of bone in contact for components made from Comparative Example 1 and Example 6 materials.

Granules comprising the material of Example 6 may advantageously be used to produce blocks as described herein.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of making a prosthetic dental item, the method comprising:
(i) selecting a block having a square or rectangular cross-section along is extent, wherein said block comprises a polymeric material which comprises a repeat unit of formula (I): 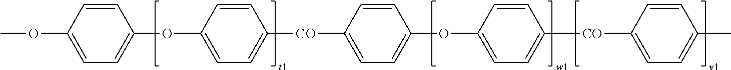
where t1 and w1 independently represent 0 or 1 and v1 represents 0, 1 or 2;
wherein said polymeric material has a crystallinity of at least 10%;
(ii) machining the block in dependence upon data collated using digital technology.

2. A method according to claim 1, wherein the crystallinity of said polymeric material is greater than 20%, preferably greater than 30%.

3. A method according to claim 1, wherein said block has a volume of at least 900 mm³ and less than 65,000 mm³; and/or said block has a minimum side length of 7 mm and a maximum of 75 mm; and/or said block has a cross-section with an area in the range 50 mm² to 1000 mm² (preferably 50 to 200 mm²); and/or said block has a length in the range 10 mm to 80 mm (preferably 15 mm to 20 mm).

4. A method according to claim 1, wherein said polymeric material consists essentially of a repeat unit of formula I, wherein t1=1, v1=0 and w1=0.

5. A method according to claim 1, wherein said polymeric material has a melt viscosity in the range 0.09 to 0.5 KNsm⁻².

6. A method according to claim 1, wherein said composition comprises at least 80 wt % or at least 94 wt % of said polymeric material.

7. A method according to claim 1, wherein said block comprises a composition comprising said polymeric material (especially a polymeric material consisting essentially of repeat units of formula I wherein t1=1, w1=0 and v1=0) and an apatite, wherein the ratio of the wt % of said polymeric material divided by the wt % of said apatite is in the range 1 to 9.

8. A method according to claim 7, wherein the ratio of the wt % of said polymeric material divided by the wt % of said apatite is in the range 3.8 to 4.2.

9. A method according to claim 7, wherein said composition includes at least 65 wt % of said polymeric material and includes at least 10 wt % of said apatite; and the sum of the wt % of said polymeric material and said apatite is in the range 90 to 100 wt %.

10. A method according to claim 7, wherein said apatite is a hydroxyapatite.

11. A method according to claim 7, wherein the D50 of said apatite, assessed using laser diffraction and based on a volume distribution, is less than 200 μm, preferably less than 20 μm.

12. A method according to claim 1, wherein, in the method, in a step (i)* after step (i) and before step (ii), digital technology is used to collate data on the region into which the dental item is to fit; and wherein step (i)* includes scanning a region into which the dental item is to fit or scanning of a model of a region into which the dental item is to fit.

13. A method according to claim 1, wherein the ratio of the crystallinity (measured by DSC) of the block selected in step (i) divided by the crystallinity (measured by DSC) of the dental item produced in the method is in the range 0.8 to 1.2, preferably in the range 0.9 to 1.1,

14. A method according to claim 1, wherein said dental item made in the method includes an area of at least 0.5 cm², preferably at least 1 cm², which has a thickness of less than 1.0 mm.

15. A method according to claim 1, wherein said dental item is selected from an inlay, onlay, crown, bridge, dental implant or abutment.

16. A method according to claim 1, wherein said dental item is made from said block which comprises said polymeric material and an apatite.

17. A dental item made in the method of claim 1.

18. A dental item according to claim 17, wherein said dental item is selected from an inlay, onlay, crown, bridge, dental implant or abutment, wherein the crystallinity of said polymeric material is greater than 20% (preferably greater than 30%), wherein said polymeric material consists essentially of a repeat unit of formula I, wherein t1=1, v1=0 and w1=0; wherein said polymeric material has a melt viscosity in the range 0.09 to 0.5 KNsm⁻², wherein said dental item includes an area of at least 0.5 cm² (preferably at least 1 cm²) which has a thickness of less than 1.0 mm. wherein, optionally, said dental item includes an apatite, wherein the ratio of the wt % of said polymeric material divided by the wt % of said apatite is in the range 1 to 9 (preferably in the range 3.8 to 4.2).

19. A block for use in the method of claim 1, said block consisting essentially of a composition which includes:
(a) at least 99 wt % of a polymeric material wherein said polymeric material comprises a repeat unit of formula (I) 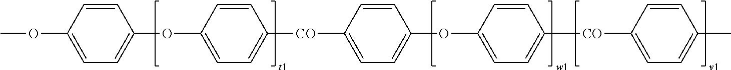
wherein t1=1, w1=0 and v1=0, wherein said polymeric material has a crystallinity
measured by DSC of at least 10% (preferably 20 to 36%); or
(b) said polymeric material of formula I and an apatite, wherein t1=1, w1=0 and v1=0, wherein said polymeric material has a crystallinity measured by DSC of at least 10% (preferably 20 to 36%); wherein the ratio of the wt % of said polymeric material
divided by the wt % of said apatite is in the range 1. to 9.
